(19) United States
(12) Patent Application Publication
(10) Pub. No.: US 2025/0339154 A1
(43) Pub. Date: Nov. 6, 2025

(54) Left Atrial Appendage Occluder

(71) Applicant: St. Jude Medical, Cardiology Division, Inc., St. Paul, MN (US)

(72) Inventors: Brian Joseph Perszyk, Shoreview, MN (US); Andrey Kai, Plymouth, MN (US)

(73) Assignee: St. Jude Medical, Cardiology Division, Inc., St. Paul, MN (US)

(21) Appl. No.: 19/182,718

(22) Filed: Apr. 18, 2025

Related U.S. Application Data
(60) Provisional application No. 63/642,973, filed on May 6, 2024.

(51) Int. Cl. A61B 17/12; A61B 17/00

(52) U.S. Cl. CPC A61B 17/12172; A61B 2017/00477; A61B 17/12122

(57) ABSTRACT

A collapsible and expandable medical device may include a proximal end disc having a diameter that is greater than a distal lobe of the medical device. The distal lobe may have an axial length. The proximal disc may be connected to the distal lobe by a connecting member. A stabilizing wire may be coupled to the distal lobe, and may have a backing portion, and first and second legs terminating in first and second hooks, respectively, that are configured to engage tissue at a target implant site. The stabilizing wire may have an axial length measured from a proximal-most end of the backing portion to a distal-most end of the first and second hooks when the medical device is in the expanded condition, the axial length of the stabilizing wire being between about one quarter and about one half of the axial length of the lobe.

### CROSS-REFERENCE TO RELATED APPLICATIONS

This application claims the benefit of the filing date of U.S. Provisional Patent Application No. 63/642,973, filed May 6, 2024, the disclosure of which is hereby incorporated by reference herein.

### FIELD OF THE DISCLOSURE

The present disclosure relates generally to medical devices that are used in the human body. In particular, the present disclosure is directed to an occlusion device having a configuration that allows for more consistent and stable anchoring of the occlusion device within a tissue cavity. More specifically, the present disclosure is directed to an occlusion device with one or more rows of hooks or stabilizing wires that increase the resilience of the device to embolization or motion in the implanted condition.

### BACKGROUND

An occluder is a medical device used to treat (e.g., occlude) tissue at a target site within the human body, such as an abnormality, a vessel, an organ, an opening, a chamber, a channel, a hole, a cavity, a lumen, or the like. For example, an occluder may be used for Left Atrial Appendage (“LAA”) closures. An LAA is a normal anatomical structure in which there is a sac in the muscle wall of the left atrium. When a patient experiences atrial fibrillation (“AFib”), a blood clot may be formed within the LAA which may become dislodged and enter into the blood stream. By occluding the LAA, the release of blood clots from the LAA may be significantly reduced, if not eliminated. Various techniques have been developed to occlude the LAA. For instance, balloon-like devices have been developed that are configured to be implanted completely within the cavity of the LAA, while surgical techniques have also been developed where the cavity of the LAA is inverted and surgically closed.

Despite these techniques, it would be advantageous to provide an improved occlusion device that offers a reduced risk of adverse events such as embolization.

### SUMMARY OF THE DISCLOSURE

According to one aspect of the disclosure, a collapsible and expandible medical device is for treating a target site. The medical device may include a proximal end comprising a disc defining a diameter in an expanded condition of the medical device, and a distal end comprising a lobe defining a diameter and an axial length in the expanded condition of the medical device, the diameter of the disc being larger than the diameter of the lobe. The medical device may also include a connecting member connecting the disc to the lobe, and a stabilizing wire coupled to the lobe, the stabilizing wire having a backing portion, a first leg extending from the backing portion, a second leg extending from the backing portion, the first leg terminating in a first hook, the second leg terminating in a second hook, the first and second hooks being configured to engage tissue at the target site. The stabilizing wire may have an axial length measured from a proximal-most end of the backing portion to a distal-most end of the first and second hooks when the medical device is in the expanded condition, the axial length of the stabilizing wire being between about one quarter and about one half of the axial length of the lobe. The lobe may be formed as a braid including a first strand extending in a first direction and a second strand extending in a second direction, the first direction being angled relative to the second direction. The first leg may be oriented substantially along the first direction, and the second leg may be oriented substantially along the second direction. The first direction may be angled about 90 degrees relative to the second direction. The first hook may be substantially parallel to the first direction, and the second hook may be substantially parallel to the second direction. The stabilizing wire may further include a first straight segment between the first leg and the first hook, and a second straight segment between the second leg and the second hook, the first straight segment and the second straight segment being substantially parallel to a central longitudinal axis of the medical device so that the first hook and the second hook are also substantially parallel to the central longitudinal axis of the medical device. The proximal-most end of the backing portion of the stabilizing wire may be positioned radially outside of the lobe, and a majority of the first leg and a majority of the second leg may each be positioned radially inside of the lobe. The proximal-most end of the backing portion of the stabilizing wire may be positioned radially inside of the lobe, and a majority of the first leg and a majority of the second leg may each be positioned radially outside of the lobe. The stabilizing wire may include a plurality of stabilizing wires. The plurality of stabilizing wires may include a first group of stabilizing wires and a second group of stabilizing wires, the first group of stabilizing wires being positioned on the lobe proximally of the second group of stabilizing wires. Each stabilizing wire of the first group may be positioned between a circumferentially adjacent pair of stabilizing wires of the second group, and each stabilizing wire of the second group may be positioned between a circumferentially adjacent pair of stabilizing wires of the first group. The lobe may include a proximal portion defining a proximal surface of the lobe, a distal portion defining a distal surface of the lobe, and a middle portion connecting and extending between the proximal portion and the distal portion, and a first transition between the proximal portion and the middle portion may be curved and a second transition between the middle portion and the distal portion may be curved. The first transition may have a radius of curvature of between about 0.04 inches (about 1.016 mm) and about 0.06 inches (about 1.524 mm). The second transition may have a radius of curvature of between about 0.04 inches (about 1.016 mm) and about 0.06 inches (about 1.524 mm). In the expanded condition of the medical device, the lobe may be outwardly bowed such that the middle portion of the lobe extends farther radially outwardly of a central longitudinal axis of the medical device than do the distal surface of the lobe and the proximal surface of the lobe.

According to another aspect of the disclosure, a collapsible and expandible medical device is for treating a target site, and the medical device may include a proximal end comprising a disc defining a diameter in an expanded condition of the medical device, a distal end comprising a lobe defining a diameter in the expanded condition of the medical device, the diameter of the disc being larger than the diameter of the lobe, a connecting member connecting the disc to the lobe, and a stabilizing wire coupled to the lobe, the stabilizing wire being configured to engage tissue at the target site. A distal surface of the disc may include a plurality of barbs, hooks, or tines configured to frictionally engage tissue defining an ostium of the target site. The distal surface of the disc may include the plurality of hooks, and each of the plurality of hooks may be separately coupled to the disc. The distal surface of the disc may include the plurality of tines, and the disc may be formed of a plurality of strands of wires braided together, at least some of the plurality of strands having free ends, the free ends of the at least some of the plurality of strands defining the plurality of tines. The distal surface of the disc may include the plurality of barbs, and the plurality of barbs may be formed on a suture that is coupled to the disc. The medical device may include a patch of fabric within the disc, and the patch of fabric may be coupled to the disc by the suture.

According to a further aspect of the disclosure, a collapsible and expandible medical device is for treating a target site, and the medical device may include a proximal end comprising a disc defining a diameter in an expanded condition of the medical device, a distal end comprising a lobe defining a diameter in the expanded condition of the medical device, the diameter of the disc being larger than the diameter of the lobe, a connecting member connecting the disc to the lobe, and a stabilizing wire coupled to the lobe, the stabilizing wire being configured to engage tissue at the target site. The disc may be formed of a plurality strands of wire braided together, and free ends of the plurality of strands may be gathered and secured within a retaining cap. The retaining cap may include an internally threaded end screw and a marker band. The end screw may extend proximally from a proximal surface of the disc. A transition member may extend from the proximal surface of the disc to the end screw to create a smooth transition between the disc and the end screw. The transition member may be formed as a spray-coated polymer. The transition member may be formed of a fabric. The fabric may have a second end coupled to the end screw, and a first end coupled to the disc at a location radially inward of an outer periphery of the disc. The fabric may have a second end coupled to the end screw, and a first end coupled to the disc at a location at or adjacent to an outer periphery of the disc. The fabric may be the only fabric that is coupled to the disc.

According to still another aspect of the disclosure, a collapsible and expandible medical device is for treating a target site. The medical device may include a proximal end comprising a disc defining a diameter in an expanded condition of the medical device, a distal end comprising a lobe defining a diameter in the expanded condition of the medical device, the diameter of the disc being larger than the diameter of the lobe, a connecting member connecting the disc to the lobe, and a stabilizing wire coupled to the lobe, the stabilizing wire being configured to engage tissue at the target site. The disc may have a first stiffness, and the lobe may have a second stiffness different than the first stiffness. The lobe may be formed non-integrally with the disc. The lobe may be formed from braided wires having an average diameter that is different than an average diameter of braided wires forming the disc. The lobe may be formed from braided wires having an austenite transformation finish temperature that is different than an austenite transformation finish temperature of braided wires forming the disc. The lobe may be formed integrally with the disc. The lobe and/or the disc may be formed with two layers of braided wires. The two layers of braided wires may include an outer layer and an inner layer, the outer layer being softer than the inner layer. The outer layer may have a diameter that is larger than a diameter of the inner layer such that there is open space between the outer layer and the inner layer.

### BRIEF DESCRIPTION OF THE DRAWINGS

FIG. 1 illustrates a known medical device.

FIGS. 2A-2C are a schematic diagram of the known medical device shown in FIG. 1 under radial compression.

FIG. 3 is a schematic diagram of a delivery system in accordance with the present disclosure.

FIG. 4 illustrates a side view of an exemplary embodiment of a medical device including a rounded lobe.

FIGS. 5A-B illustrate the medical device of FIG. 4 in an exemplary low use range at or near maximum compression, and in an exemplary high use range at or near minimum compression, respectively.

FIG. 6 illustrates an alternative version of the medical device of FIG. 4.

FIGS. 7A-B illustrate the medical device of FIG. 6 in an exemplary low use range at or near maximum compression, and in an exemplary high use range at or near minimum compression, respectively.

FIG. 8A illustrates the medical device of FIG. 4 compressed within a tube representing a body cavity.

FIG. 8B illustrates an alternative version of the medical device of FIG. 4 compressed within a tube representing a body cavity.

FIG. 9A illustrates a highly schematic view of a stabilizing wire of the medical device of FIG. 4.

FIGS. 9B-D illustrate highly schematic views of other embodiments of stabilizing wires that may be used as an alternative to that shown in FIG. 9A.

FIG. 9E illustrates the medical device of FIG. 4 that incorporates two rows of the stabilizing wires of FIGS. 9B-C.

FIGS. 10A-B illustrate examples of a medical device with different examples of friction-enhancing features on a disc thereof.

FIG. 11A illustrates an example of a proximal end cap on a disc of a medical device.

FIG. 11B illustrates another example of a proximal end cap on a disc of a medical device.

FIG. 12 is an enlarged view of a stabilizing wire coupled to a lobe of a medical device.

### DETAILED DESCRIPTION OF THE DISCLOSURE

The present disclosure relates generally to medical devices that are used in the human body. Specifically, the present disclosure provides for various features that may be incorporated into a left atrial appendage occluder for improved functionality. It is contemplated, however, that the described features and methods of the present disclosure as described herein may be incorporated into any number of systems as would be appreciated by one of ordinary skill in the art based on the disclosure herein.

Although the exemplary embodiment of the medical device is described as treating a target site including a LAA, it is understood that the use of the term “target site” is not meant to be limiting, as the medical device may be configured to treat any target site, such as an abnormality, a vessel, an organ, an opening, a chamber, a channel, a hole, a cavity, or the like, located anywhere in the body. The term “vascular abnormality,” as used herein is not meant to be limiting, as the medical device may be configured to bridge or otherwise support a variety of vascular abnormalities. For example, the vascular abnormality could be any abnormality that affects the shape of the native lumen, such as an atrial septal defect, a lesion, a vessel dissection, or a tumor. Embodiments of the medical device may be useful, for example, for occluding a patent foramen ovalis (“PFO”), atrial septal defect (“ASD”), ventricular septal defect (“VSD”), or patent ductus arteriosus (“PDA”), as noted above. Furthermore, the term “lumen” is also not meant to be limiting, as the vascular abnormality may reside in a variety of locations within the vasculature, such as a vessel, an artery, a vein, a passageway, an organ, a cavity, or the like. As used herein, the term “proximal” refers to a part of the medical device or the delivery device that is closest to the operator, and the term “distal” refers to a part of the medical device or the delivery device that is farther from the operator at any given time as the medical device is being delivered through the delivery device. In addition, the terms “deployed” and “implanted” may be used interchangeably herein.

Some embodiments of the present disclosure provide an improved percutaneous catheter directed intravascular occlusion device for use in the vasculature in patients' bodies, such as blood vessels, channels, lumens, a hole through tissue, cavities, and the like, such as a LAA. Other physiologic conditions in the body occur where it is also desirous to occlude a vessel or other passageway to prevent blood flow into or therethrough. These device embodiments may be used anywhere in the vasculature where the anatomical conditions are appropriate for the design.

The medical device may include one or more layers of occlusive material, wherein each layer may be comprised of any material that is configured to substantially preclude or occlude the flow of blood so as to facilitate thrombosis. As used herein, “substantially preclude or occlude flow” shall mean, functionally, that blood flow may occur for a short time, but that the body's clotting mechanism or protein or other body deposits on the occlusive material results in occlusion or flow stoppage after this initial time period.

Some embodiments of the present disclosure may be formed by a plurality of wire strands having a predetermined relative orientation with respect to one another. However, it is understood that according to additional embodiments of the present disclosure, that the medical device could be etched or laser cut from a tube, or the device could comprise an occlusion material coupled to a scaffolding structure or a plurality of slices of a tubular member coupled together.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

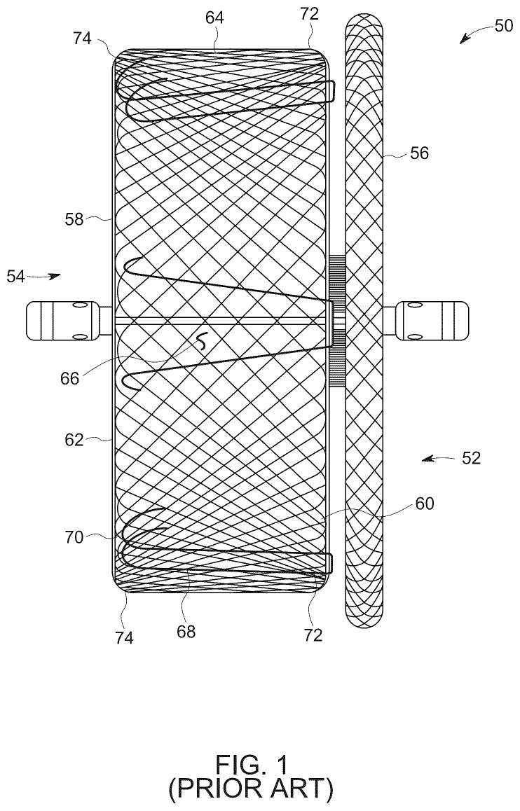

In at least some conventional or known medical devices used for the occlusion of abnormalities, such as a medical device 50 shown in FIG. 1, medical device 50 includes a proximal end 52 and a distal end 54, with a disc 56 at proximal end 52 and a lobe 58 at distal end 54. The lobe 58 has a proximal edge 60 (also referred to as a proximal face), a distal edge 62 (also referred to as a distal face), and a middle or central portion 64 that defines a cavity 66. The medical device 50 also includes stabilizing wires 68 secured to a radially outer or circumferential surface of middle portion 64. The stabilizing wires 68 terminate in a hook 70 at free ends thereof, and thereby facilitate retention of the medical device 50 at a target site and preventing the medical device 50 from becoming dislodged from the target site after deployment.

In this known medical device 50, proximal edge 60 and distal edge 62 adjoin middle portion 64 at a first relatively blunt or sharp (e.g., non-rounded) transition 72 and a second blunt transition 74, respectively. First blunt transition 72 connects proximal edge 60 to middle portion 64 by an approximately 90 degree angle. Likewise, second blunt transition 74 connects distal edge 62 to middle portion 64 by an approximately 90 degree angle. First blunt transition 72 and second blunt transition 74 partially define a generally rectangular cross section to lobe 58, leading to relatively blunt circumferential edges of the device and relatively high radial force applied to the surrounding tissue.

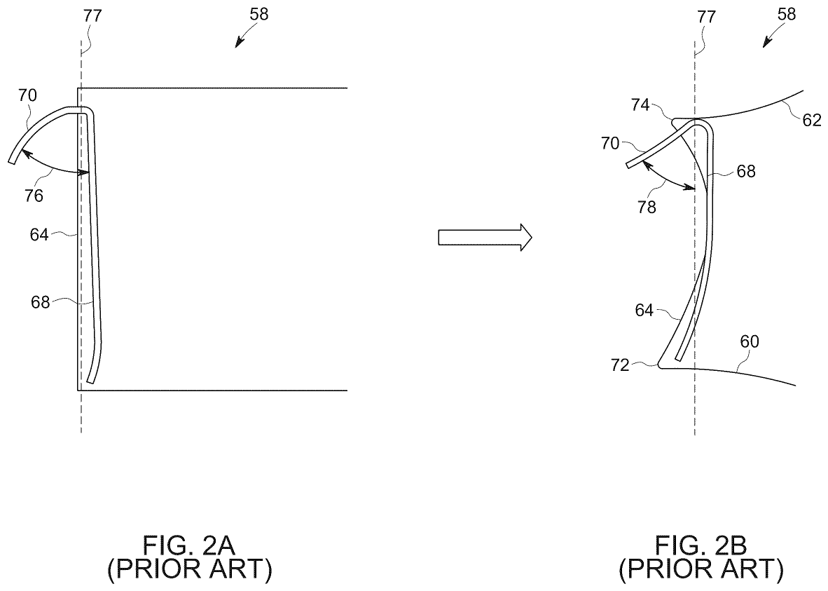

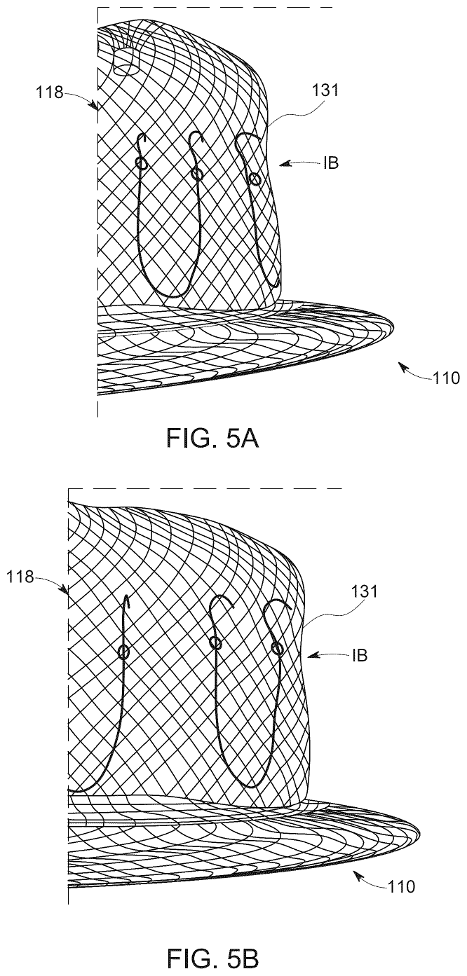

Turning now to FIGS. 2A-2C, medical device 50 before and after undergoing radial compression is depicted. Before radial compression (FIG. 2A) is applied to lobe 58, the outer surface of middle portion 64 is linear or extends generally perpendicular to proximal and distal faces 60, 62. Each hook 70 of a corresponding stabilizing wire 68 is at a first angle 76 with respect to a generally longitudinal direction 77. When radial compression is applied to lobe 58 (FIGS. 2B and 2C), proximal and distal faces 60, 62 flex and bow outwardly (e.g., axially outward), and middle portion 64 of lobe 58 flexes and bows inwardly, in response to the applied force. The approximately 90 degree angle of first blunt transition 72 and second blunt transition 74 force the outer surface of middle portion 64 to transition from linear to concave when proximal and distal faces 60, 62 bow outwardly. The concave shape adopted by lobe 58 also shifts the position of stabilizing wires 68, such that stabilizing wires 68 at least partially contract and hooks 70 transition from first angle 76 to a second, greater angle 78. At second angle 78, hooks 70 are oriented more directly towards the adjacent tissue, than when hooks 70 are at first angle 76. This shift in orientation of stabilizing wires 68, and therefore hooks 70, can lead to an increase in interactions between hooks 70 and the adjacent tissue at the target site within the patient's body. The increased interaction with tissue can lead to adverse side effects such as late pericardial effusion. In various embodiments described herein, the lobe may have a generally cylindrical shape (e.g. with a substantially circular-cross section), although it should be understood that the proximal and distal ends of the lobe may have various types of curvature described herein, and some embodiments may include a bowed cylindrical shape for the lobe, for example as described in greater detail in connection with FIG. 6 below.

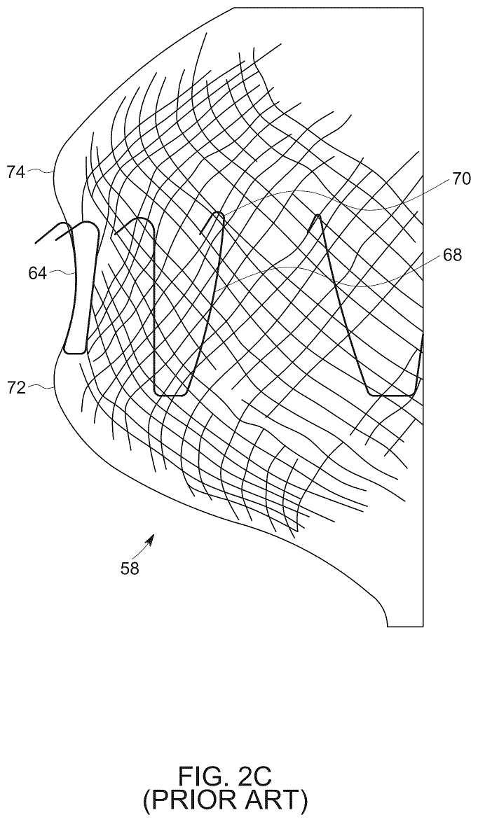

Turning now to FIG. 3, a schematic diagram of a delivery system 100 is shown. Delivery system 100 includes a delivery device 102 including a catheter 104 and a coupling member 106 configured to couple a distal end of a delivery cable 108 to a medical device 110 (which may be any of the occluders described herein) for facilitating the deployment of medical device 110 at a target site. Medical device 110 is deployed to treat the target site, and, in the example embodiment, is an occlusion device (“occluder”).

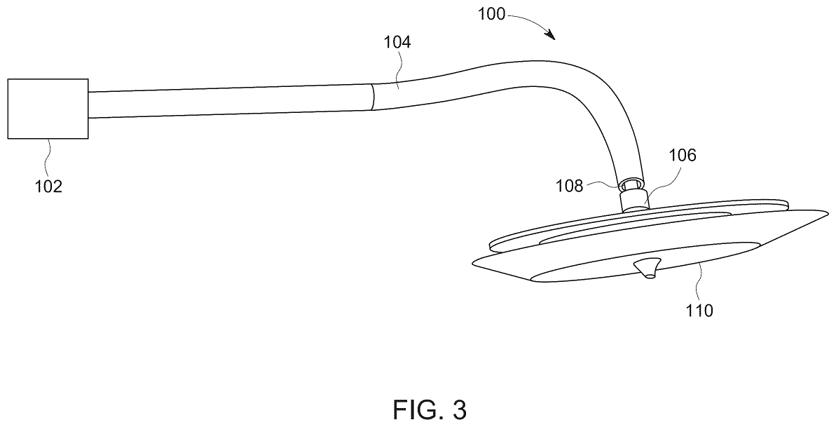

FIG. 4 illustrates an exemplary embodiment of medical device 110. Medical device 110 includes a proximal end 112 and a distal end 114. Proximal end 112 includes a disc 116 (which may be generally similar to disc 56), and distal end 114 includes a lobe 118 (which may be generally similar to lobe 58), wherein disc 116 and lobe 118 are connected by a connecting member 120 (which may be generally similar to the corresponding structure in medical device 50). Lobe 118 includes a proximal portion 122 defining a proximal surface 124 of lobe 118, a distal portion 126 defining a distal surface 128 of lobe 118, and a middle or central portion 130 connecting and extending between proximal portion 122 and distal portion 126. Central portion 130 has a circumferential or radially outer surface 131. Proximal surface 124 is connected to or adjoins middle portion 130 at a first transition T1, and distal surface 128 is connected to or adjoins middle portion 130 at a second transition T2. First transition T1 may be generally similar to the first blunt transition 72 of medical device 50, for example having a radius of curvature of about between 0 mm (e.g., a right angle) and about 2 mm. Second transition T2 may be significantly more rounded, and have for example a radius of curvature of about 0.1 inches (about 2.54 mm).

Lobe 118 may further include a plurality of stabilizing wires 132 coupled to lobe 118 at radially outer surface 131 (also referred to as circumferential surface) of central or middle portion 130. Stabilizing wires 132 may each include a hook 134 at a terminal end thereof. Hooks 134 extend radially outward from middle portion 130 of lobe 118.

Some embodiments of medical device 110 may be formed from a braided fabric or mesh material including a plurality of wire strands having a predetermined relative orientation with respect to one another. However, it is understood that according to additional embodiments of the present disclosure, medical device 110 could be etched or laser cut from a tube, or the device could comprise an occlusion material coupled to a scaffolding structure or frame.

In one embodiment, medical device 110 is formed from a shape-memory material including a metal fabric. The metal fabric is deformed to generally conform to a surface of a mandrel. While on the surface of the mandrel, the metal fabric is treated under heated conditions to allow for the heat-setting of the metal fabric. The heat-setting of the metal fabric ensures that the metal fabric will retain the substantial shape of the mandrel once it is removed from the surface of the mandrel. In the exemplary embodiment, the mandrel utilized for the heat-setting treatment defines the radii of curvature adopted by the metal fabric for the edges of lobe 118 of medical device 110, specifically first transition T1 and second transition T2.

The radius of curvature selected and defined for second transition T2 may round or soften the circumferential edges of medical device 110. This rounding or softening of the circumferential edge leads to a reduction in the radial force applied to the surrounding tissue. Therefore, medical device 110 is more conformable to the anatomy of the target site in which it is deployed, specifically an LAA.

Disc 116 of medical device 110 is configured to abut the adjacent wall surrounding the opening of the vascular defect to prevent movement of medical device 110 and to assist in sealing of the abnormality in which medical device 110 is deployed. Different sizes and shapes of the disc are contemplated. In one embodiment, the disc portion may be larger in diameter than the vascular abnormality to be occluded to be capable of overlying the opening of the abnormality.

Lobe 118 of medical device 110 is formed to have a suitable size to engage with the lumen of the abnormality that is to be occluded. Medical device 110 may then be held at the target site by radial engagement between lobe 118 and the lumen of the abnormality. Hooks 134 of stabilizing wires 132 also engage with the surrounding tissue and improve retention of medical device 110 at the target site.

One particular shape memory material that may be used to form medical device 110 (and, particularly, lobe 118) as described herein is Nitinol. Nitinol alloys are highly elastic and are said to be “superelastic,” or “pseudoelastic.” This elasticity may allow medical device 110 to be resilient and return to a preset, expanded configuration for deployment following passage in a distorted form through delivery catheter 104. Further examples of materials and manufacturing methods for medical devices with shape memory properties are provided in U.S. Patent Application Publication No. 2007/0265656 titled “Multi-layer Braided Structures for Occluding Vascular Defects” and filed on Jun. 21, 2007, which is incorporated by reference herein in its entirety.

It is also understood that medical device 110 may be formed from various materials other than Nitinol that have elastic properties, such as stainless steel, trade named alloys such as Elgiloy®, or Hastalloy, Phynox®, MP35N, CoCrMo alloys, metal, polymers, or a mixture of metal(s) and polymer(s). Suitable polymers may include PET (Dacron), polyester, polypropylene, polyethylene, HDPE, Pebax, nylon, polyurethane, silicone, PTFE, polyolefins and ePTFE. Additionally, it is contemplated that the medical device may comprise any material that has the desired elastic properties to ensure that the device may be deployed, function as an occluder as disclosed within this application.

Referring still to FIG. 4, although it may be beneficial to achieve higher conformity of the lobe 118, the radius of curvature of about 0.1 inches at the second transition T2 may have a negative result. For example, as shown in FIG. 4, the hooks 134 are generally positioned within the area of the second transition T2. The ability of the hook 134 to anchor into tissue may be affected depending on the precise location of each hook 134 relative to the specific area of the second transition T2. This variability may be an undesirable result of such a large radius at the second transition T2. Thus, in some embodiments, the radius of curvature at the second transition T2 may be reduced to between about 0.03 inches (about 0.762 mm) and about 0.08 inches (about 2.032 mm), or in some embodiments between about 0.04 inches (about 1.016 mm) and about 0.06 inches (about 1.524 mm), and in other embodiments about 0.05 inches (about 1.27 mm). It has been found that such a reduction in the radius of curvature of second transition T2 still maintains most or all of the benefit of the increase conformability of the second transition T2 having a radius of curvature of about 0.1 inches, while significantly reducing the anchoring sensitivity resulting from the precise location of the hook 134 on the lobe 118. It should be understood that, in some embodiments, the first transition T1 may have a radius of curvature within the same range as provided above for the second transition T2 (e.g., between about 0.03 inches and about 0.08 inches).

Although the embodiment of medical device 110 shown and described in connection with FIG. 4 includes one group of similar or identical stabilizing wires 132 with hooks 134 positioned at or near the second transition T2, in some embodiments, it may be preferable to include a second set of stabilizing wires with “shorter” hooks. For example, between each pair of stabilizing wires 132, a smaller stabilizing wire with a pair of hooks may be positioned near a mid-point between the proximal surface 124 and the distal surface 128 of the lobe 118. Various options for these types of hooks are described in more detail in U.S. Patent Application No. 63/562,341, filed Mar. 7, 2024 and titled “Left Atrial Appendage Occluder Devices,” the disclosure of which is hereby incorporated herein. In these embodiments, the shorter stabilizing wires may be generally intended to grip tissue at a different location than the stabilizing wires 132 shown in FIG. 4. For example, if the medical device 110 is for use in occluding the LAA, the shorter stabilizing wires may be configured to engage tissue near the entrance into the LAA, for example just distal to the location of the circumflex artery.

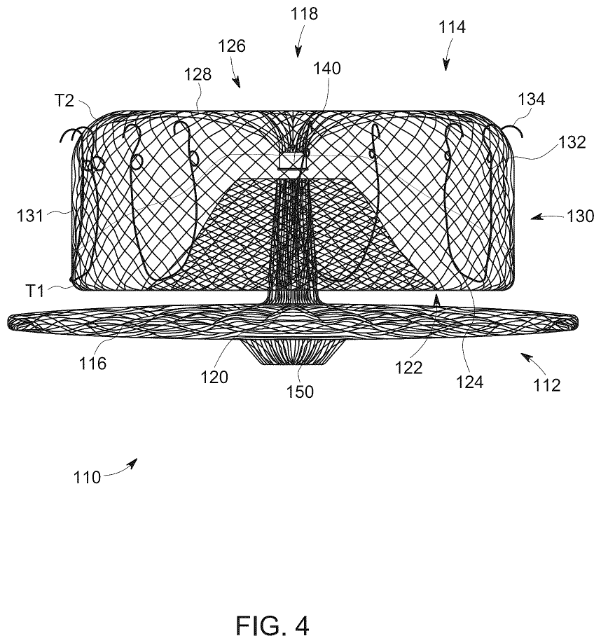

FIGS. 5A-B illustrate the medical device 110 of FIG. 4 in an exemplary low use range at or near maximum compression, and high use range at or near minimum compression, respectively. In other words, in FIGS. 5A-B, the medical device 110 is shown as if the lobe 118 is in an implanted condition and in contact with tissue such that the lobe 118 is under compression, but the environment (e.g. the tissue compressing the lobe 118) is omitted from FIGS. 5A-B to more clearly show the medical device 110. It should be understood that, typically, medical device 110 may be provided in multiple different sizes intended for different anatomical size ranges. In one example, medical device 110 may be provided in a small size that has a lobe 118 with a length of about 7.5 mm, and in a large size that has a lobe 118 with a length of about 10 mm. Each of the small and large size medical devices 110 may be provided with different diameters of the lobe 118. For example, the small size medical device 110 may be provided in options that have lobes 118 with diameters of 16 mm, 18 mm, 20 mm, or 22 mm, while the large size medical device may be provided in options that have lobes 118 with diameters of 25 mm, 28 mm, 31 mm, or 34 mm.

Still referring to FIGS. 5A-B, it can be seen that when medical device 110 is implanted at a target site within the low use range at or near maximum compression (shown in FIG. 5A) or within the high use range, at or near minimum compression (shown in FIG. 5B), the radially outer surface 131 of the middle or central portion 130 of the lobe 118 may undergo inward bowing IB. It should be understood that the representative target site of FIG. 5A is relatively small (resulting in higher compression) compared to the representative target site of FIG. 5B (resulting in relatively lower compression). For any hooks of stabilizing wires that are positioned near the point in the lobe 118 at which the inward bowing IB occurs, the hooks may not be able to effectively engage with tissue because the hooks may also be pulled radially inwardly, making it more difficult for the hooks to contact tissue surrounding the lobe 118. It should be understood that the portion of the lobe 118 showing the inward bowing IB is not in direct contact with the surrounding tissue (which, as noted above, is omitted from the views of FIGS. 5A-B), and thus the inward bowing IB results in the lobe 118 being spaced away from the nearby tissue at the location of the inward bowing IB.

FIG. 6 illustrates a medical device 110a in an unconstrained or expanded condition, which may be an alternate version of the medical device 110 of FIG. 4. Medical device 110a may be substantially similar or identical to medical device 110, with certain exceptions noted below. For example, medical device 110a may include a disc 116 connected to a lobe 118a, with the lobe 118a including a proximal face 124, a distal face 128, and a middle or central portion 130a that defines a circumferential or radially outer surface 131a. The main difference between medical device 110a and medical device 110 is that the shape of the lobe 118a of medical device 110a, compared to the lobe 118 of medical device 110, is modified by bowing or rounding the radially outer surface 131a of the lobe 118a outwardly. For example rather than having a generally linear radially outer surface 131 extending between the proximal face 124 and distal face 128 of lobe 118, lobe 118a may include a bowed outer surface 131a between the proximal face 124 and distal face 128 in which, in the proximal-to-distal direction, the radius or diameter of the lobe 118a first increases to a maximum at or near an axial mid-point between the proximal face 124 and distal face 128, and then decreases from the axial mid-point to the distal face 128. In some examples, the radius or diameter of the lobe 118a at the proximal face 124 is about equal to the radius or diameter of the lobe 118a at the distal face 128, although in other embodiments, they may be unequal with either one being larger than the other, while still being smaller than the maximum radius or diameter at the peak of the outwardly bowed portion. In some examples, this outer bowing in the lobe 118a may be achieved via shape-setting (e.g. by applying heat while the lobe 118a is over a suitably-shaped mandrel).

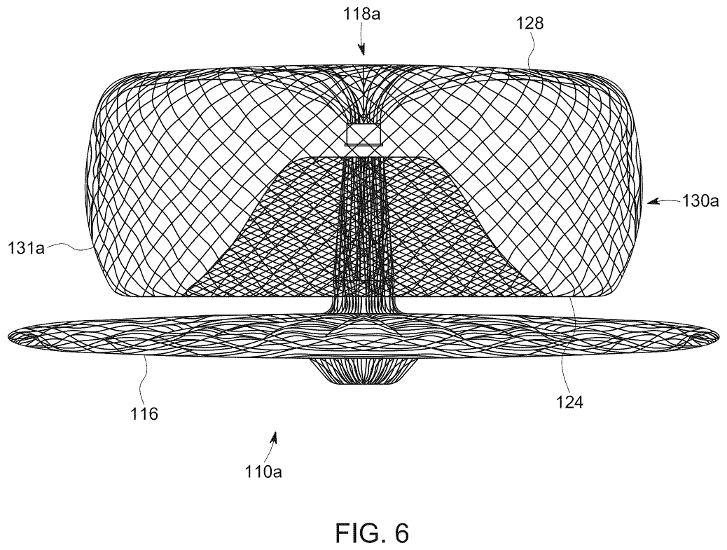

In the example of FIG. 6, by providing the lobe 118a with an outwardly bowed surface, when the medical device 110a is placed under compression, the tendency to bow inwardly as shown and described in connection with FIGS. 5A-B may be resisted. For example, FIGS. 7A-B illustrate the medical device of FIG. 6 in an exemplary low use range at or near maximum compression, and in an exemplary high use range at or near minimum compression, respectively. Like in FIGS. 5A-B, in FIGS. 7A-B, the medical device 110a is shown as if the lobe 118a is in an implanted condition and in contact with tissue such that the lobe 118a is under compression, but the environment (e.g. the tissue compressing the lobe 118a) is omitted from FIGS. 7A-B to more clearly show the medical device 110a. The implanted/deployed conditions shown in FIGS. 7A-B with medical device 110a correspond to the respective implanted/deployed conditions shown in FIGS. 5A-B with medical device 110. As can be seen by comparing FIGS. 5A-B with FIGS. 7A-B, the outward bowing provided in medical device 110a helps to counteract the tendency of the lobe 118a to bow inwardly under compression, resulting in a substantially linear or straight radially outer surface 131a of the lobe 118a when under compression. In other words, in FIGS. 5A-B, the inward bowing IB of lobe 118 results in an inward divot in the side wall of the lobe 118, whereas under the same implantation conditions, the lobe 118a of medical device 110a avoids having inward bowing like that shown in FIGS. 5A-B. This may result in better engagement between hooks of stabilizing wires on lobe 118a with surrounding tissue.

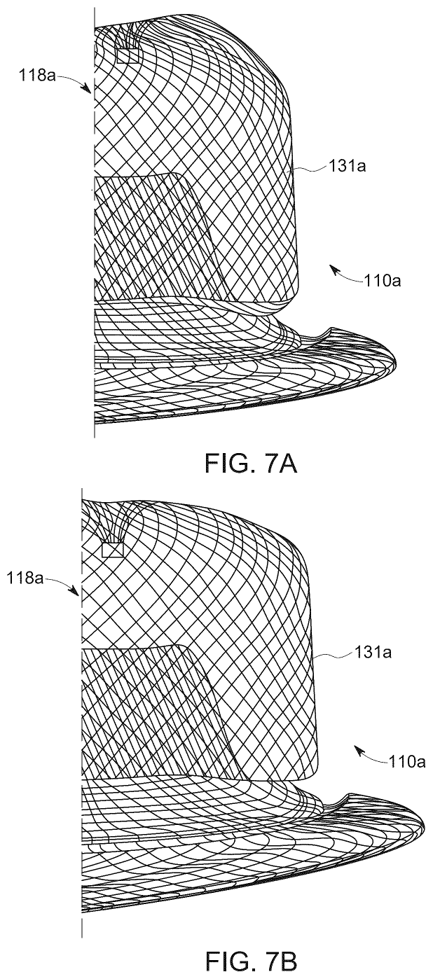

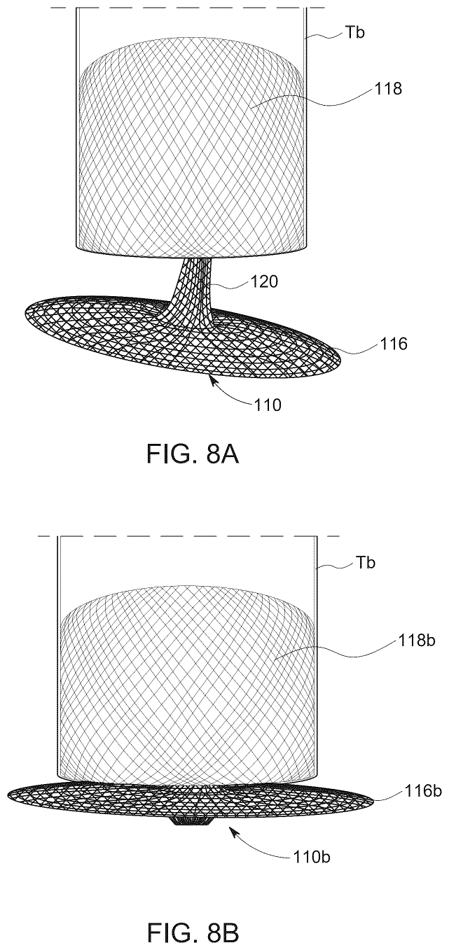

FIG. 8A illustrates medical device 110 of FIG. 4 with the lobe 118 thereof compressed within a tube Tb representing a body cavity, such as the LAA. The relatively blunt transition T1 may provide relatively high radial forces on the tissue receiving the lobe 118, which is desirable. However, the blunt transition T1 may also tend to force the disc 116 to jump or push out of the lobe 118, a condition illustrated in FIG. 8A. As shown in FIG. 8A, the connecting member 120, which is normally mostly or fully nested within the lobe 118 is extending proximally from the lobe 118 such that the disc 116 does not make good contact with the opening of the tube Tb, which may represent the ostium of the LAA. It would be desirable for the disc 116 to be positioned against, or even pulled against, the ostium of the LAA (represented by the opening of the tube Tb), to help ensure a good seal at the opening.

One option for reducing the tendency for the disc 116 to “jump” away from the lobe 118 is to provide a larger radius at the transition T1 so that it is not as blunt. For example, in one embodiment, the transition T1 was rounded to have a radius of curvature of about 0.1 inches. This relatively large transition reduced the radial force which the proximal lobe applied to surrounding structure/tissue, which may be undesirable. However, in another embodiment, shown in FIG. 8B, medical device 110b was provided with a lobe 118b having a proximal transition T1 of about 0.05 inches. Other than this proximal transition T1 being larger than that of medical device 110, medical device 110b may be identical to medical device 110. This increase of the proximal transition T1 (compared to medical device 110) to about 0.05 inches, without making the proximal transition T1 larger (e.g., 0.1 inches or above), the proximal lobe 118b was still able to provide a suitable amount of radial force, similar to medical device 110, but the rounded proximal transition T1 avoided the tendency of the disc 116b to jump or push proximally relative to the lobe 118b. As shown in FIG. 8B, the disc 116b of medical device 110b is pulled tightly against the opening of the tube Tb, which may represent the ostium of the LAA.

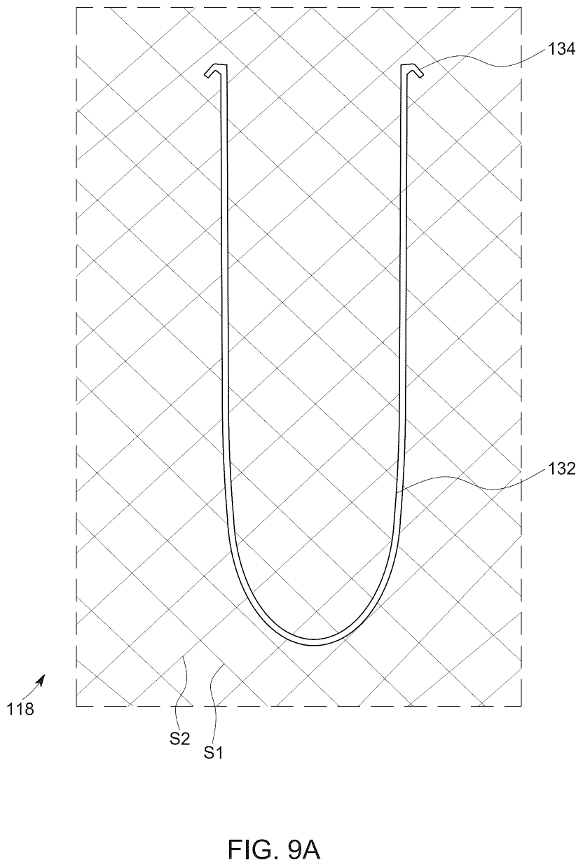

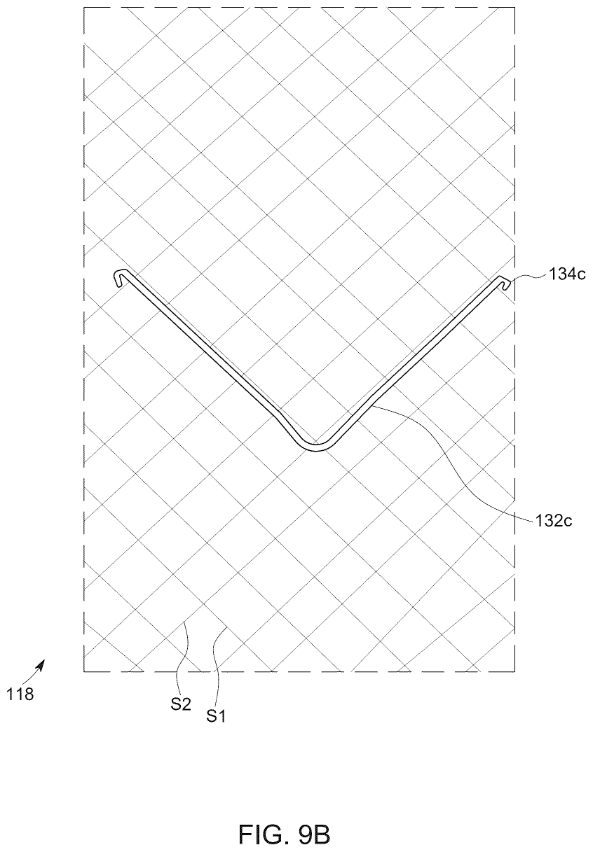

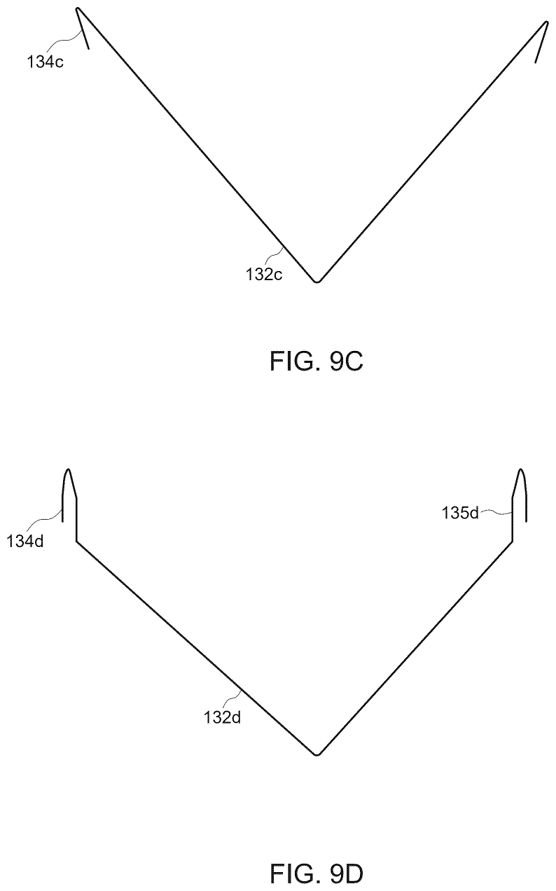

FIG. 9A illustrates a highly schematic view of a stabilizing wire 132 of the medical device 110 of FIG. 4. Referring to FIGS. 4 and 9A, each stabilizing wire 132 may have a rounded backing portion (which may also be referred to as an apex), positioned near the proximal surface 124 of the lobe 118 (toward the top of the view of FIG. 9A). Two legs may extend from the backing portion, with the two legs extending nearly the entire axial length of the lobe 118, until each of the two legs transitions into a hook 134 positioned near the distal surface 128 of the lobe 118. With this configuration, each stabilizing wire 132 may extend an axial length that is between about 80% and about 100% of the axial length of the lobe 118 when the lobe 118 is in an unbiased condition. This may result in significant braid elongation relative to the length of the stabilizing wire 132 during collapsing of the lobe 118. In some examples, stabilizing wires 132 include an eyelet on one or both legs to allow for coupling the stabilizing wire 132 to the lobe 118 via sutures or similar string-like members. If the stabilizing wires 132 are sutured to the lobe 118 via such eyelets, there is a risk of the braid of the lobe 118 deforming due to the braid elongation.

FIG. 9A schematically illustrates that the braided wire(s) forming lobe 118 may include one or more strands, generally including a first group of strands S1 that are interwoven or interlaced with a second group of strands S2, with the strands S1 and the strands S2 being oriented at an angle relative to each other, which may be generally about 90 degrees in the unbiased condition of the lobe 118 (although other angles besides 90 degrees may be suitable). As can be seen in FIG. 9A, the legs of the stabilizing wire 132 generally do not follow the directionality of either of the two groups of strands S1, S2. This may result in movement of the stabilizing wire tip (e.g., hook 134) relative to the strands S1, S2 forming the braid. Such movement may increase the risk of the hook 134 catching behind the braid during expansion of the lobe 118, which may prevent the hook 134 from returning to its optimal position upon deployment of the lobe 118 into the target site.

FIGS. 9B-C illustrate highly schematic views of another embodiment of a stabilizing wire 132c that may be used as an alternative to that shown in FIG. 9A. FIG. 9B shows the stabilizing wire 132c within the same lobe 118 shown in FIG. 9A, while FIG. 9C shows the stabilizing wire 132c in isolation. Stabilizing wire 132c may be formed in the same way as stabilizing wire 132, with the exception being the shape and size. For example, stabilizing wire 132c may include a backing portion (which may also be referred to as an apex), and two legs extending from the backing portion, with the two legs each terminating in a hook 134c. There are two main differences between stabilizing wire 132c and stabilizing wire 132. First, the overall axial height (measured in the direction of the lobe 118, from the apex or backing portion to the hook 134c), is smaller than that of stabilizing wire 132. For example, whereas stabilizing wire 132 may extend an axial length that is between about 80% and about 100% of the axial length of the lobe 118 when the lobe 118 is in an unbiased condition, stabilizing wire 132c may extend an axial length that is between about 25% and about 50% of the axial length of the lobe 118 when the lobe 118 is in an unbiased condition. Second, the two legs of stabilizing wire 132c are spread more widely (e.g., at a greater angle) compared to the two legs of stabilizing wire 132. In some embodiments, the two legs of the stabilizing wire 132c, when the lobe 118 is in the unbiased condition, may be between about 40 degrees and about 110 degrees, including about 80 degrees and about 100 degrees, including about 90 degrees. When the stabilizing wire 132c is attached to the lobe 118, each leg of the stabilizing wire 132c may closely follow the directionality of a corresponding strand S1, S2. With this configuration, as the lobe 118 is collapsed and the braid elongates, the stabilizing wire may generally follow the elongation of the braid as strands S1 change angle relative to strands S2. This may result in a reduction in inconsistency associated with using longer stabilizing wires (e.g., stabilizing wire 132 shown in FIG. 9A) that do not follow (or do not closely follow) the braid of the lobe 118. It should be understood that these benefits may be achieved even if the legs of the stabilizing wire 132c do not perfectly follow the directionality of the corresponding strands S1, S2, but rather generally or substantially follow the direction of the strands. Another benefit of using the stabilizing wire 132c shown in FIGS. 9B-C is that, because the stabilizing wire is axially shorter, two (or more) separate rows of stabilizing wires may be connected to the lobe 118. For example, the stabilizing wire 132 of FIG. 9A extends nearly the entire axial length of the lobe 118, meaning that there may not be room for additional stabilizing wires positioned on the lobe 118 distal or proximal of the long stabilizing wire 132. And even if there was room for an additional stabilizing wire, an additional stabilizing wire may in that scenario require an overlap of the materials that increases the collapsed profile of the device, increasing forces required during use and also increasing the required size of the delivery system, which are both generally not desirable outcomes. However, with stabilizing wire 132c, the relatively short axial length of the stabilizing wire 132c means that one row of stabilizing wires 132c may be positioned on the proximal portion of the lobe 118 while another row of stabilizing wires 132c may be positioned on the distal portion of the lobe 118 (with potentially additional row(s) of stabilizing wires between these two rows). The use of multiple rows of stabilizing wires 132c may provide for additional locations at which the hooks 134c may engage tissue, which may increase the stability (and/or decrease the likelihood of embolization) of the medical device 110 that incorporates the multi-row stabilizing wire 132c configuration. Another potential benefit of the use of shorter stabilizing wires 132c having a wider leg angle is that the stabilizing wires 132c may be able to be more evenly spaced circumferentially compared to shorter stabilizing wires that have substantially parallel legs. As used herein, when it is described that a leg of a stabilizing wire follows the directionality of a corresponding braid strand, it may be preferable for there to be less than a 60 degree difference between the directionalities, preferably less than a 30 degree difference between the directionalities.

Referring still to FIG. 9C, in one embodiment, the hooks 134c of stabilizing wire 132c extend at an angle that is substantially parallel to the angle of the corresponding legs of the stabilization wire 132c. With this configuration, the hooks 134c may be oriented at an angle relative to the direction in which a pulling force may be applied when the hooks 134c are engaged with tissue. In other words, upon implantation, it may be desirable for the medical device 110 to resist being pulled proximally relative to the tissue with the pulling force directed substantially parallel to the central longitudinal axis of the medical device 110. However, with stabilizing wires 132c shown in FIG. 9C, the hooks 134c may be oriented at an angle relative to this expected pulling force (e.g., oblique to the central longitudinal axis of the medical device 110). In some embodiments, it may be desirable to align the directionality of the hook with the pulling force. Thus, as shown in FIG. 9D, another embodiment of a stabilizing wire 132d is identical to stabilizing wire 132c except for the shape of the hook 134d. As shown in FIG. 9D, the hooks 134d may be oriented at an oblique angle relative to the legs of the stabilizing wire 132d from which the hooks 134d extend, while being parallel to the central longitudinal axis of the medical device 110 (and thus substantially parallel to an expected directionality of pulling force that the stabilizing wires 132d are configured to help resist). In some embodiments, a short straight section 135d may be provided at the transition between the leg of the stabilizing wire 132d and the hook 134d of the stabilizing wire, which may help ensure the hook 134d is engaging tissue in the same direction in which it is resisting embolization. If such a straight section 135d is provided, the transition from the leg of the stabilizing wire 132d to the straight section 135d may act as a suitable location to couple (e.g., via sutures) the stabilizing wire 132d to the strands S1, S2 of the lobe 118, and may help prevent the stabilizing wire 132d from being pulled through the braid. In other embodiments, instead of providing straight section 135d at the transition between the leg and the hook 134d, a straight section may be provided on the hook 134d itself, or the hook 134d may gradually curve so that at least the tip of the hook 134d is oriented substantially parallel to the central longitudinal axis of the medical device 110.

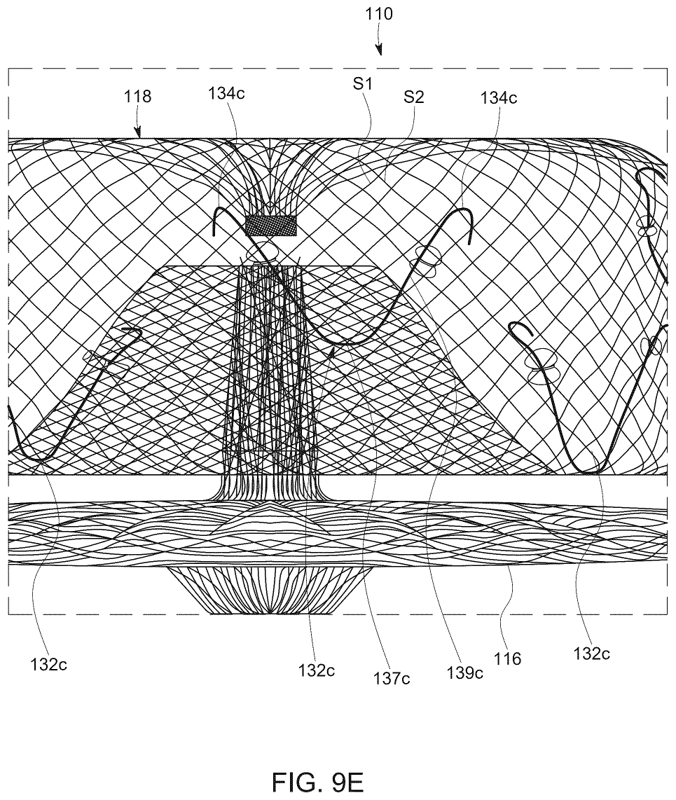

FIG. 9E illustrates medical device 110 that incorporates two rows of the stabilizing wires 134c of FIGS. 9B-C, with one row of stabilizing wires 132c positioned distally and one row positioned proximally. In the illustrated embodiment, each proximal stabilizing wire 132c is positioned between a pair of circumferentially adjacent distal stabilizing wires 132c, and each distal stabilizing wire 132c is positioned between a pair of circumferentially adjacent proximal stabilizing wires 132c. As described above, these stabilizing wires 132c are relatively short with wider set hooks 134d, which may allow for symmetric circumferential spacing, substantially following strand/braid wires of the lobe 118 for a more repeatable and/or predictable loading of the medical device 110 into a delivery device, and deployment of the medical device 110 from the delivery device, with less stress on the stabilizing wires 132c, sutures that connected the stabilizing wires 132c to the lobe 118, and strands of the braid that interact with the stabilizing wires 132c. It should be understood that, although the stabilizing wire 132c of FIG. 9B is shown as having legs that exactly follow corresponding strands S1, S2, the wide-set version of stabilizing wires 132c does not need to have legs that exactly follow corresponding strands S1, S2. For example, the stabilizing wires 132c shown in FIGS. 9E and 10A-10B have legs that extend between (or across) about two adjacent (and substantially parallel) corresponding strands S1, S2.

Any of the stabilizing wires described herein may be formed in any suitable fashion. U.S. Patent Application Publication No. 2022/0280166, the disclosure of which is hereby incorporated by reference herein, describes various suitable stabilizing wires that may be used with the devices described herein. In one example, the stabilizing wires may be formed by laser cutting the stabilizing wires from a sheet of material (e.g., from a flat sheet of nitinol), and the then subsequently electropolishing and shape-setting the stabilizing wires. However, there may be one or more disadvantages of forming the stabilizing wires via laser cutting and subsequent electropolishing. For example, small defects resulting from the laser cutting and/or electropolishing process may lead to inconsistencies in manufacturing quality, or even increased likelihood of damage due to fatigue. The laser cutting process may also create heat affected zones, which can also increase the likelihood of damage due to fatigue. Other concerns include difficulty in sourcing raw materials, resulting sharp edges that can abrade sutures, and increased manufacturing costs due to complex manufacturing processes.

One alternative option to using laser cutting with subsequent electropolishing is using round wire as the initial material to form the stabilizing wires. Round wires are relatively easy to manipulate and to process, do not pose any fatigue concerns, do not have sharp edges that can damage sutures, and are readily available for sourcing. One potential disadvantage of forming the stabilizing wires from round wires is that it may be difficult to form an eyelet in the stabilizing wire to receive a suture for attachment. However, particularly for shorter stabilizing wires with a relatively short axial length between the apex and hook (e.g., as shown in FIGS. 9B-D), the eyelet may be entirely omitted without hindering the ability to suture or otherwise connect the stabilizing wires to the braid of the lobe. However, even if it is desirable to form an eyelet-like feature in a stabilizing wire formed from a round wire, a small jog or wave can be added to the round wire to perform the same function as the eyelet. When forming the stabilizing wires from round wires, the wires may be cut to the desired length using snips or another similar cutting tool, which in some cases may result in a jagged tip end of the round wire. To mitigate concerns of any jagged tip ends (which could for example cause damage to the sheath of a delivery device), the tips can be laser cut and/or laser welded to round the tips to mitigate jagged tip ends. The stabilizing wires 132c shown coupled to lobe 118 in FIG. 9E are formed from round wires, instead of laser cutting with subsequent electropolishing.

For all of the embodiments of medical devices described above, it may be preferable for the disc (e.g., disc 116) to be softer than in prior art devices. For example, if the disc is softer, it may be able to conform more readily to the anatomy. This enhanced conformability may help to prevent the disc from “falling” off of the coumadin ridge (a muscular ridge of tissue between the left superior pulmonary vein and the LAA) into the LAA. Situations in which the disc is pulled into the LAA may be associated with higher thrombosis and stroke rates. In addition to softening the disc, softening the lobe (e.g., lobe 118 or any other lobe described herein) can increase the conformability of the lobe to the anatomy, which may prevent situations in which the lobe deforms the anatomy which might cause damage such as perforations to the tissue.

As used herein, “softness” refers generally to a deformability of a material or structure. The softer a material is, the more readily it will deform when engaged with adjacent tissue. Softness may, in some instances, be contrasted with “stiffness,” which refers generally to a resistance to deformation. The stiffer a material or structure is, the more it will resist deformation when engaged with adjacent tissue. Accordingly, where a “softness” of a first structure is contrasted with a “stiffness” (“less softness”) of a second structure, this description may refer generally to an increased deformability of the first structure as compared to a decreased deformability of the second structure.

There are various ways in which the disc and/or lobe may be constructed, processed, and/or modified to have an increased level of softness and thus increased deformability. In one example, the disc can be softened by electropolishing the braid wires at the outer edges of the disc. In another example, the disc can be softened by grit blasting the braid wires at the outer edges of the disc. In some examples, when softening the disc, it may be desirable to soften only the outer edge(s) of the disc because the outer edge(s) of the disc are likely to be in contact with tissue, while the radially inner areas are likely to avoid contact with tissue, and it may be desirable to maintain axial tension between the disc and the lobe to help secure the disc against tissue.

In another example, the lobe and/or disc may be softened by using a softer braid wire to form the lobe and/or disc. In other examples, a soft lobe and/or disc may be achieved using a modified heat treatment process. Further details of these examples are provided below.

In some examples, if it is desirable to form the disc as having a softness that is different than the softness of the lobe, the softness of the lobe and braid can be made different by either (i) heat treating the lobe using a different method than the heat treatment used for the disc; and/or (ii) forming the lobe and the disc as two separate (i.e., non-integral) members having different properties, and then coupling the lobe to the disc to form the medical device. For example, when the occluder is formed of strands of nitinol, adjusting the temperature experienced by the nitinol during heat treatment can affect the stiffness of the material, as well as the austenite transformation finish temperature. Compared to typical treatments, either overheating or underheating the nitinol (or certain areas or section of the nitinol) may soften the nitinol to help achieve a variable softness. In examples where the lobe and disc formed as two separate members that each include wire strands (e.g., nitinol wire strands), the wire strands of the disc may be provided with smaller diameter or thickness compared to the wire strands that form the lobe. For example, the wires forming the disc may have an average diameter that is smaller, compared to the average diameter of the wires forming the lobe, by about 0.00025 inches (about 0.00635 mm). For example, all of the wires that form the disc may be smaller in diameter by about 0.00025 inches than all of the wires that form the lobe, or half of the wires that form the disc may be smaller in diameter than the wires that form the lobe by about 0.0005 inches (about 0.0127 mm), with the other half of the wires that form the disc being about the same diameter as the wires that form the lobe. In some examples, referring to average differences, the average diameter of the wires forming the disc may be smaller than the average diameter of the wires forming the lobe by an amount between about 0.00025 inches (about 0.00635 mm) and about 0.002 inches (0.0508 mm), including between about 0.0005 inches (0.0127 mm) and about 0.001 inches (0.0254 mm).

In some examples, the softness of the disc and lobe can be formed differently by utilizing two separate layers of braid within the medical device, with the first braid layer having a first softness extending out to the disc and the second braid layer having a second softness (which may be different than the first softness) extending partially outward in the disc, and one or both layers extending outward in the lobe.

U.S. Patent Application Publication No. 2023/0404559, the disclosure of which is hereby incorporated by reference herein, provides various methods for creating an occluder device having different levels of softness for enhanced conformability. It should be understood that the methodology described within the '559 Publication may be applied to LAA occluders, including medical device 110 (and variants thereof described herein). For example, the '559 Publication describes the use of two braid layers with different softness to form a disc, similar to the disc of the occluder(s) described herein. However, similar or the same concepts may be applied in forming the lobe of the occluder(s) described herein. For example, when applying these concepts to the lobe, two braid layers could be provided on top of each other so that the layers could each be about the same diameter as the lobe, or in other embodiments, a softer outer braid layer may be provided with a diameter that is larger than the diameter of a less soft inner braid layer, which may allow for more conformability and/or more tissue contact with the outer layer, while maintaining radial strength from the inner layer. This feature may be important when the occluder has a relatively large oversizing for the target tissue (e.g. the LAA). It should be understood that these approaches may be combined with other embodiments described herein, such as the bowed shape of the lobe 130a of the medical device 110a of FIG. 6.

As noted above, if the lobe is formed to be softer, and thus more comfortable, it may be able to conform to the anatomy better than less soft (e.g., stiffer) lobes. This may, at least in part, allow for more oversizing with a given medical device. In other words, even if the lobe is oversized for the target site (e.g., the cavity within the LAA), the oversized lobe may be tolerated better because it will more effectively conform to the target site. This enhanced tolerance of the anatomy to the size of the lobe may limit the total number of device sizes needed to treat a wide patient population, since target sites vary in size (and shape) among the population. This would also help with being able to create multiple disc sizes per lobe size for optimal sealing at the ostium without needing to have an unmanageable number of total device size combinations.

It should be understood that, although different options for increasing softness are described above, one or more of those options may be used in combination, and if the softness of both the disc and the lobe are increased, the increase in softness may be achieved in either the same way for the disc and the lobe, or in different ways.

For any of the embodiments of the medical devices above, it may be preferable for the disc (e.g., disc 116) to have an enhanced gripping feature, which may provide an enhanced level of frictional engagement with, or “stickiness” to, tissue that the disc engages. For example, by providing the disc with enhanced gripping features, the disc may be more likely to maintain contact with the coumadin ridge and less likely to “slip” or be pulled into the LAA. By maintaining contact between the disc and the coumadin ridge, the medical device may provide for optimal sealing of the LAA (and, correspondingly, stroke reduction). One or more features may be used to provide such enhanced grip. In one example, very small hooks or tines may be attached, e.g. via sutures, to the distal face of the disc (which is the face that, in use, confronts the ostium of the LAA. If such hooks or tines are provided, in some examples they may be limited to being positioned at or near the outer radius of the disc, since that is the portion expected to be in contact with the tissue forming the ostium of the LAA). FIG. 10A illustrates one example of a medical device 110 with these types of hooks H on the disc 116. In other examples, the strands that form the braid of the medical device and/or the disc may be used to enhance frictional engagement. For example, one or more additional strands may be added to the braid, with the additional strands being intentionally cut (or otherwise provided with a free end), with the cut or free ends of the additional strand(s) extending outwardly and/or distally to engage tissue forming the ostium of the LAA. This approach may also be used for the stabilizing wires of the lobe. In another embodiment, two wire strands may be run per braid carrier, with one of the two wire strands being stopped short in the braid (or otherwise cut out of the braid after braiding). This approach may result in a braid that is similar to a typical braid, except that it has extra wire strands running next to some of the braid wires that are subsequently formed into hooks. This particular approach may allow for the use of different size wire stands for these hook wires only, and avoid the need to separately couple hooks (e.g. by suturing) into the braid frame. In some or all of the medical device embodiments above, a fabric or other patch of material is provided within the disc in order to help enhance sealing upon implantation of the medical device. In embodiments with such a patch, it may be coupled to the disc in various different ways, such as by suturing the patch to the strands of the braid that forms the disc. The particular suture material chosen to perform such suturing may be selected to be a high-friction suture material. For example, sutures with barbs, such as Quill Barbed Sutures offered by Corza Medical, Inc., may be used to couple the fabric patch to the disc. In some examples, the sutures with enhanced gripping features may be provided on only the distal portion of the disc. FIG. 10B illustrates one example of a medical device 110 with this type of barbed suture BS on the disc 116, although it should be understood that FIG. 10B omits the fabric patch positioned within the disc 116 for clarity of illustration. It should be understood that, although various individual options are provided above for enhancing the grip of the disc with the tissue forming the LAA ostium, one or more of these options may be combined in a single device.

Referring briefly again to FIG. 4, in some embodiments, the medical device 110 includes one or more features into which ends of the strands of the wires forming the braid may be gathered and/or secured. For example, in FIG. 4, a distal retainer or end cap 140 is positioned within the lobe 118, with ends of the strands that form the lobe 118 hooking proximally and being secured to and within the end cap 140. In this embodiment, the distal end cap 140 is inverted in the sense that it is positioned within the lobe 118 and proximally to the distal face 128 of the lobe 118. A proximal retainer or end cap 150 may provide a generally similar function on the proximal portion of the disc 116, although in some embodiments the proximal end cap 150 may also include an internal thread. For example, the proximal end cap 150 in FIG. 4 may be the component to which the coupling member 106 of the delivery system 100 of FIG. 3 temporarily couples to during delivery of the medical device 110. Additional details of suitable configurations of proximal end cap 150 are described in U.S. Pat. No. 8,758,389, the disclosure of which is hereby incorporated by reference herein.

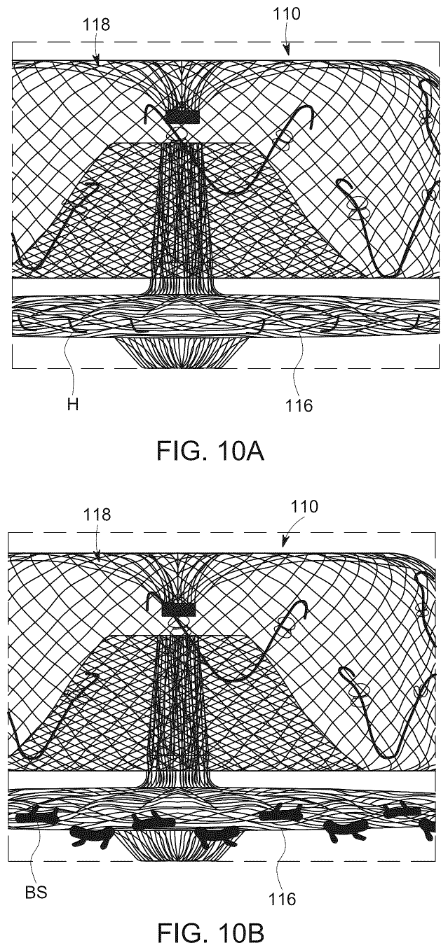

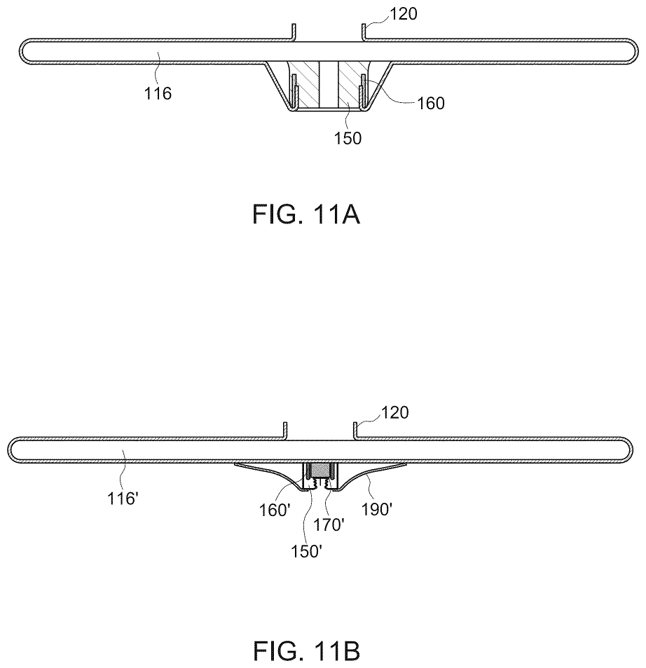

FIG. 11A illustrates one example of the proximal end cap 150, which may also be referred to as an end screw. The disc 116 and a portion of the connecting member 120 are shown in FIG. 11A, with other components of the medical device 110 omitted for clarity. The end screw 150 may have an interior channel with threads that are complementary to threads of a component of the delivery system 100. The end screw 150 may also include one or more pockets or recesses between the interior channel and the exterior surface of the end screw 150, so that ends of strands that form the braid of the disc 116 may be wrapped or tucked into that space of the end screw 150. With this configuration, strands of the braid form a proximally-extending ramped surface between the proximal face of the disc 116 and the proximal end of the end screw 150. In some embodiments, either in addition to the recesses or pockets of the end screw 150, or instead of such recesses or pockets, a radiopaque marker band 160 may be positioned around or within the end screw 150, which may help with visualization (e.g., under fluoroscopy) of the medical device 110 during implantation.

Although the design of the proximal end screw 150 (and marker band 160, if included) is suitable for many uses, the particular stack-up of components may limit how small the medical device 110 may be collapsed, and thus limit how small the delivery system 100 may be, with the understanding that smaller delivery systems, all else being equal, are generally preferable over larger delivery systems when it comes to intravascularly delivered medical devices. FIG. 11B illustrates an alternative configuration to that shown in FIG. 11A. For example, FIG. 11B illustrates a disc 116′ and a portion of the connecting member 120, with other components of the medical device omitted for clarity. It should be understood that, other than the interaction between the disc 116′ and the end screw, the disc 116′ may be similar or identical to disc 116. For example, the proximal face of the disc 116′ may be substantially planar, with ends of the strands of the braid forming the disc being positioned in a gathered section 170′, around which a marker band 160′ may be positioned. The end cap or end screw 150′ may partially or completely surround the marker band 160′ and the gathered section 170′ of braid strands, and may include an internal channel with internal threads for interaction with external threads of a portion of delivery system 100.

One of the main differences between the configurations of FIGS. 11A and 11B is that, in the configuration of FIG. 11A, the ends of the braid strands travel proximally and then invert to travel again distally to be secured to end screw 150, whereas the ends of the braid strands in FIG. 11B travel a shorter distance to gathered section 170′ where they are secured. Further, in the configuration of FIG. 11B, a fabric portion 190′ extends from a proximal face of the disc 116′ to the end screw 150′. This fabric portion 190′ may help eliminate any recesses where blood may tend to stagnate, may provide a smooth transition on the proximal face of the medical device, and/or may improve endothelial response (e.g. tissue ingrowth) across the center of the device compared to a similar device lacking the fabric.

Still referring to the embodiment of FIG. 11B, the fabric 190′ has a first end coupled to the disc 116′ and a second end coupled to the end screw 150′. In some embodiments, like the particular one shown in FIG. 11B, the first end of the fabric 190′ may be coupled so that it only spans a short distance of the disc 116′ relatively close to the center where the end screw 150′ is positioned. In other embodiments, the first end of the fabric 190′ may extend all the way to the outer edge of the disc 116′ so that the fabric 190′ spans all or substantially all along the proximal disc 116′. In this example in which the fabric 190′ covers most or all of the disc 116′, a separate sealing patch or separate occluding fabric (e.g., positioned within the disc 116′) may be omitted. Although the embodiments described above in connection with FIG. 11B refer to a fabric 190′, it should be understood that other materials may take the place of fabric 190′, for example a spray coated polymer transition between the disc 116′ and the end screw 150′. Compared to the embodiment of FIG. 11A, the embodiment of FIG. 11B (and alternative options described therewith) may have an overall lower profile, with the end screw 150′ extending a shorter distance away from the disc 116′, which may help the medical device achieve an overall lower profile when being delivered, although the functionality of the end screw 150′ (and related components) shown and described in connection with FIG. 11B may be substantially identical to the functionality of the end screw 150 (and related components) shown and described in connection with FIG. 11A. In some examples, the lower profile may be achieved, at least in part, by shortening the end screw 150′ and/or the marker band 160′. In some examples, the lower profile may be achieved, at least in part, by avoiding the braid having to fold back on itself (as in the embodiment of FIG. 11A) and avoiding the stacking of the marker band on top of the end screw.

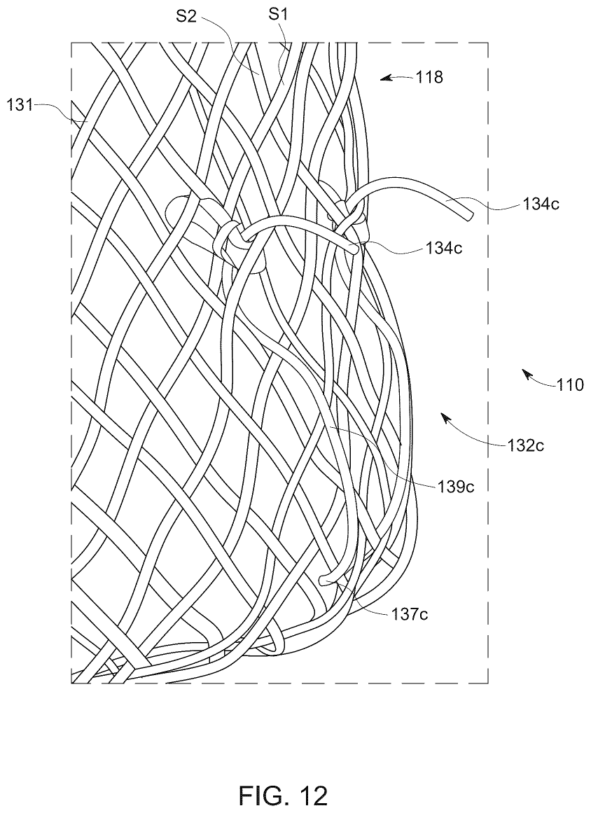

FIG. 12 is an enlarged view of a stabilizing wire 132c coupled to the lobe 118 of medical device 110. Stabilizing wire 132c of FIG. 12 may be the same as that shown in FIG. 9E, including being formed from a rounded wire, instead of being formed via laser-cutting and subsequent electropolishing. However, although the structure of the stabilizing wires 132c of FIGS. 9E and 12 may be substantially similar or identical, the way in which they interface with the lobe 118 is different. For example, in the embodiment of FIG. 9E, the apex or backing portion 137c of the stabilizing wire 132c is positioned radially outward of the outer surface 131 of the lobe 118. However, in the embodiment of FIG. 12, the apex or backing portion 137c of the stabilizing wire 132c is positioned radially inward of the outer surface 131 of the lobe 118 (and thus within the lobe 118). Further, in the embodiment of FIG. 9E, the two legs 139c that extend from the backing portion 137c almost immediately cross through an opening of the braid so that all of (or the majority of) the legs 139c are positioned radially inward of the lobe 118. However, in the embodiment of FIG. 12, the two legs 139c that extend from the backing portion 137c almost immediately cross through the opening of the braid so that all of (or the majority of) the legs 139c are positioned radially outward of the lobe 118. Referring again to FIG. 9E, the tip portions of the hooks 134c extend through an opening in the braid so that the tips are positioned exterior to the lobe 118 and available to engage tissue upon implantation. However, in FIG. 12, the point of transition between the legs 139c and hooks 134c extends radially inwardly through an opening in the braid into the lobe 118, and the hook 134c then extends radially outwardly through another opening in the braid out of the lobe 118 so that the tips of the hooks 134c are available to engage tissue upon implantation. In both the embodiments of FIGS. 9F and 12, the stabilizing wires 132c are attached to strands of the braid of the lobe 118 via sutures positioned at or near the transition between the legs 139c and hooks 134c, although the connection in the embodiment of FIG. 12 may in some examples be sutured nearer the tips of the hooks 134c compared to in FIG. 9.

The way in which stabilizing wires 68 of prior art medical device 50 may be positioned relative to the interior and exterior of the braid of the lobe 58 may be generally similar to that described above for stabilizing wires 132c relative to lobe 118 shown in FIG. 9E. For example, stabilizing wire 68 is relatively simple and planar (excluding the contours of the hooks 70). Configuring the stabilizing wire 68 to enter into the interior space of the lobe 58 through the braid near proximal face 60 of the lobe 58 and then exit to the exterior of the lobe 58 through the braid near the distal face 62 of the lobe 58 may provide some amount of stability of the stabilizing wire 68 (compared to the majority of the stabilizing wire 68 being positioned external to the lobe 58). Additionally, the stabilizing wires 68 may be coupled (e.g. via suturing) close to the axial midpoint of the lobe 58 so they can flex inward as they contact tissue, which would not work if the stabilizing wires 68 were positioned external to the lobe 58. For embodiments with a stabilizing wire that has a relatively short length (e.g., of the hooked end) extending radially outward from the lobe, the stabilizing wire may need to be coupled (e.g., sutured) to the lobe at a point relatively close to the hooked tip, otherwise the hooks may flex too easily and disengage with tissue too easily. Also, by coupling (e.g. by suturing) the stabilizing wires closer to their hooked end, additional inward curvature may be needed to allow for enough inward hook flexing when the medical device is being loaded into (or recaptured into) a sheath of a delivery device. These changes that are described above for shorter axial length stabilizing wires that have the general positioning relative to the inner/outer surfaces of the lobe described in connection with stabilizing wires 132c relative to lobe 118 in FIG. 9E may provide the same functionality whether the stabilizing wires are positioned mostly interior to, or mostly exterior to, the lobe 118. However, the attachment configuration between stabilizing wires 132c and lobe 118 shown in FIG. 12 may provide one or more additional benefits. For example, with the majority of the stabilizing wires 132c being positioned exterior to the braid forming the lobe 118, the braid of the lobe may provide better support for the stabilizing wires 132c. In some examples, because the externalized stabilizing wire 132c is supported by the braid, instead of relying on a suture (or other connector) to maintain its radial position, the hooks 134c achieve a more consistent protrusion and therefore more reliable engagement with the tissue. Further, the hooks 134c in this configuration may have retractability. In other words, when the medical device 110 is collapsed into a sheath of a delivery device, such as during a recapture procedure, the inner surface of the sheath (or loader) presses against the legs 139c of the stabilizing wire 132c, which forces the hooks 134c inwardly and away from the wall of the sheath and loader, which (i) reduces loading and/or recapture forces, (ii) minimizes or eliminates the potential for damage to the loader and sheath lining, and (iii) minimizes or eliminates the risk of the sheath tip prolapsing during recapture. When the medical device 110 is deployed from the sheath, the elastic material returns to the set shape and the hooks 134c again extend outwardly to engage the tissue.

It should be understood that, although different embodiments provided herein include different features, one or more of the features may be combined into a single embodiment, and the features are generally described as parts of individual embodiments above for clarity only. For example, the following features may be provided in any combination in a single embodiment of a medical device: (i) the different sizes of the first and second transitions T1 and T2 (see, e.g., FIGS. 4, 8A-B); (ii) the use of an outwardly bowed lobe 118a (see, e.g., FIGS. 6-7B); (iii) the use of short hooks that follow the strands of the lobe braid (see, e.g., FIGS. 9B-E); (iv) the use of enhanced gripping features on the disc (see, e.g., FIGS. 10A-B); (v) the use of a low profile end screw (see, e.g., FIG. 11B); (vi) the particular way in which the stabilizing wire is connected to the lobe (see, e.g., FIG. 9E compared to FIG. 12); and (vii) modifications to increase the softness of the disc and/or lobe.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A collapsible and expandible medical device for treating a target site, the medical device comprising:
a proximal end comprising a disc defining a diameter in an expanded condition of the medical device;
a distal end comprising a lobe defining a diameter and an axial length in the expanded condition of the medical device, the diameter of the disc being larger than the diameter of the lobe;
a connecting member connecting the disc to the lobe; and
a stabilizing wire coupled to the lobe, the stabilizing wire having a backing portion, a first leg extending from the backing portion, a second leg extending from the backing portion, the first leg terminating in a first hook, the second leg terminating in a second hook, the first and second hooks being configured to engage tissue at the target site,
wherein the stabilizing wire has an axial length measured from a proximal-most end of the backing portion to a distal-most end of the first and second hooks when the medical device is in the expanded condition, the axial length of the stabilizing wire being between about one quarter and about one half of the axial length of the lobe.

2. The medical device of claim 1, wherein the lobe is formed as a braid including a first strand extending in a first direction and a second strand extending in a second direction, the first direction being angled relative to the second direction.

3. The medical device of claim 2, wherein the first leg is oriented substantially along the first direction, and the second leg is oriented substantially along the second direction.

4. The medical device of claim 3, wherein the first direction is angled about 90 degrees relative to the second direction.

5. The medical device of claim 3, wherein the first hook is substantially parallel to the first direction, and the second hook is substantially parallel to the second direction.

6. The medical device of claim 3, wherein the stabilizing wire further includes a first straight segment between the first leg and the first hook, and a second straight segment between the second leg and the second hook, the first straight segment and the second straight segment being substantially parallel to a central longitudinal axis of the medical device so that the first hook and the second hook are also substantially parallel to the central longitudinal axis of the medical device.

7. The medical device of claim 3, wherein the proximal-most end of the backing portion of the stabilizing wire is positioned radially outside of the lobe, and a majority of the first leg and a majority of the second leg are each positioned radially inside of the lobe.

8. The medical device of claim 3, wherein the proximal-most end of the backing portion of the stabilizing wire is positioned radially inside of the lobe, and a majority of the first leg and a majority of the second leg are each positioned radially outside of the lobe.

9. The medical device of claim 3, wherein the stabilizing wire includes a plurality of stabilizing wires.

10. The medical device of claim 9, wherein the plurality of stabilizing wires includes a first group of stabilizing wires and a second group of stabilizing wires, the first group of stabilizing wires being positioned on the lobe proximally of the second group of stabilizing wires.

11. The medical device of claim 10, wherein each stabilizing wire of the first group is positioned between a circumferentially adjacent pair of stabilizing wires of the second group, and each stabilizing wire of the second group is positioned between a circumferentially adjacent pair of stabilizing wires of the first group.

12. The medical device of claim 1, wherein the lobe comprises a proximal portion defining a proximal surface of the lobe, a distal portion defining a distal surface of the lobe, and a middle portion connecting and extending between the proximal portion and the distal portion, a first transition between the proximal portion and the middle portion being curved and a second transition between the middle portion and the distal portion being curved.

13. The medical device of claim 12, wherein the first transition has a radius of curvature of between about 0.04 inches (about 1.016 mm) and about 0.06 inches (about 1.524 mm).

14. The medical device of claim 12, wherein the second transition has a radius of curvature of between about 0.04 inches (about 1.016 mm) and about 0.06 inches (about 1.524 mm).

15. The medical device of claim 12, wherein in the expanded condition of the medical device, the lobe is outwardly bowed such that the middle portion of the lobe extends farther radially outwardly of a central longitudinal axis of the medical device than do the distal surface of the lobe and the proximal surface of the lobe.

16. A collapsible and expandible medical device for treating a target site, the medical device comprising:
a proximal end comprising a disc defining a diameter in an expanded condition of the medical device;
a distal end comprising a lobe defining a diameter in the expanded condition of the medical device, the diameter of the disc being larger than the diameter of the lobe;
a connecting member connecting the disc to the lobe; and
a stabilizing wire coupled to the lobe, the stabilizing wire being configured to engage tissue at the target site,
wherein a distal surface of the disc includes a plurality of barbs, hooks, or tines configured to frictionally engage tissue defining an ostium of the target site.

17. The medical device of claim 16, wherein the distal surface of the disc includes the plurality of hooks, each of the plurality of hooks being separately coupled to the disc.

18. The medical device of claim 16, wherein the distal surface of the disc includes the plurality of tines, and the disc is formed of a plurality of strands of wires braided together, at least some of the plurality of strands having free ends, the free ends of the at least some of the plurality of strands defining the plurality of tines.

19. The medical device of claim 16, wherein the distal surface of the disc includes the plurality of barbs, the plurality of barbs being formed on a suture that is coupled to the disc.

20. The medical device of claim 19, wherein the medical device includes a patch of fabric within the disc, the patch of fabric being coupled to the disc by the suture.
